(19) United States
(12) Patent Application Publication
(10) Pub. No.: US 2016/0220416 A1
(43) Pub. Date: Aug. 4, 2016

(54) LASER EYE SURGERY APPARATUS

(71) Applicant: NIDEK CO., LTD., Gamagori (JP)

(72) Inventor: Muneyuki ADACHI, Gamagori-shi (JP)

(73) Assignee: NIDEK CO., LTD., Gamagori (JP)

(21) Appl. No.: 15/010,833

(22) Filed: Jan. 29, 2016

(30) Foreign Application Priority Data
Jan. 30, 2015 (JP) 2015-017123

(51) Int. Cl. A61F 9/008

(52) U.S. Cl. CPC A61F 9/0084; A61F 2009/0087; A61F 2009/00851

(57) ABSTRACT

A laser eye surgery apparatus includes: a laser device which includes a gain-switched seed light source configured to repeatedly generate seed laser pulses with a pulse width of 10 femtoseconds or greater and 1 nanosecond or less and vary repetition frequency of the seed laser pulses according to a set repetition frequency, the laser device being configured to emit laser pulses based on the seed laser pulses generated by the gain-switched seed light source; a condenser configured to condense the laser pulses in a transparent tissue of a patient's eye to cause photodisruption at concentration positions of the laser pulses in the transparent tissue; a scanner configured to scan the concentration position of each of the laser pulses; and a controller configured to control the gain-switched seed light source to change the repetition frequency of the seed laser pulses according to a scanning speed.

### CROSS REFERENCE TO RELATED APPLICATION

This application is based upon and claims the benefit of priority of Japanese Patent Application No. 2015-017123 filed on Jan. 30, 2015, the contents of which are incorporated herein by reference in its entirety.

### BACKGROUND

The present disclosure relates to a laser eye surgery apparatus in which an operator mainly treats a transparent tissue (for example, the cornea or the crystalline lens) of a patient's eye by using photodisruption caused by laser pulses.

In the related art, a technology, by which an operator treats a patient's eye by respectively concentrating laser pulses on multiple target positions in the patient's eye, and causing photodisruption in a tissue, has been known. A technology, by which the repetition frequency of laser pulses emitted to a patient's eye is changed during an operation, has also been known.

A laser device disclosed in JP-T-2013-520846 includes an oscillator, and a cavity-dumped regenerative amplifier. The oscillator generates multiple seed laser pulses (hereinafter, may also be referred to as a “seed laser pulse train”) at a constant repetition frequency. The cavity-dumped regenerative amplifier picks up and amplifies only every fifth to 20000^{th} seed laser pulses from the seed laser pulses train generated by the oscillator. The picked-up seed laser pulses are discharged from the cavity-dumped regenerative amplifier without being amplified. That is, the cavity-dumped regenerative amplifier filters out the seed laser pulse train. The laser device disclosed in JP-T-2013-520846 changes the repetition frequency of emitted laser pulses by changing the percentage of the picked-up and amplified seed laser pulses in the seed laser pulse train generated by the oscillator.

The repetition frequency of laser pulses emitted to a patient's eye is also deemed to be changed by filtering a seed laser pulse train or an amplified laser pulse train using an acoustic-optic modulator (AOM) or the like.

### SUMMARY

When a patient's eye is treated using ultrashort-pulse laser light, it is desirable that the patient's eye can be accurately treated within a short amount of time. Accordingly, a laser eye surgery apparatus is required to scan ultrashort-pulse laser light, which is emitted at a higher repetition frequency, at a higher speed. According to a method of changing the repetition frequency by filtering out seed laser pulses train in the related art, the repetition frequency of the laser pulses emitted from the laser device can be changed only to a divisor of the repetition frequency of a seed laser pulse. As a result, according to the method in the related art, the repetition frequency is changed only in a stepwise manner, and cannot be continuously (linearly) changed. According to the method in the related art, the filter-out pulses are not used for treatment, and thus, energy is wasted.

A typical object of the present disclosure is to provide a laser eye surgery apparatus in which an operator can accurately treat a patient's eye within a short amount of time by appropriately changing the repetition frequency of the laser pulses.

According to a typical aspect of the present disclosure, there is provided a laser eye surgery apparatus comprising:

a laser device which includes a gain-switched seed light source configured to repeatedly generate seed laser pulses with a pulse width of 10 femtoseconds or greater and 1 nanosecond or less and vary repetition frequency of the seed laser pulses according to a set repetition frequency, the laser device being configured to emit laser pulses based on the seed laser pulses generated by the gain-switched seed light source;

a condenser configured to condense the laser pulses emitted from the laser device in a transparent tissue of a patient's eye to cause photodisruption at concentration positions of the laser pulses in the transparent tissue;

a scanner configured to scan the concentration position of each of the laser pulses emitted from the laser device; and

a controller configured to control the gain-switched seed light source to change the repetition frequency of the seed laser pulses according to a scanning speed at which the concentration position is scanned by the scanner.

An operator can accurately treat a patient's eye within a short amount of time by appropriately changing the repetition frequency of a laser pulse from a laser eye surgery apparatus according to the aspect of the present disclosure.

### BRIEF DESCRIPTION OF THE DRAWINGS

FIG. 1 is a schematic view illustrating the configuration of a laser eye surgery apparatus 1.

FIG. 2 is a schematic view illustrating the configuration of a laser device 10.

FIG. 3 is a flowchart illustrating a repetition frequency changing process executed by the laser eye surgery apparatus 1.

### DESCRIPTION OF ILLUSTRATIVE EMBODIMENTS

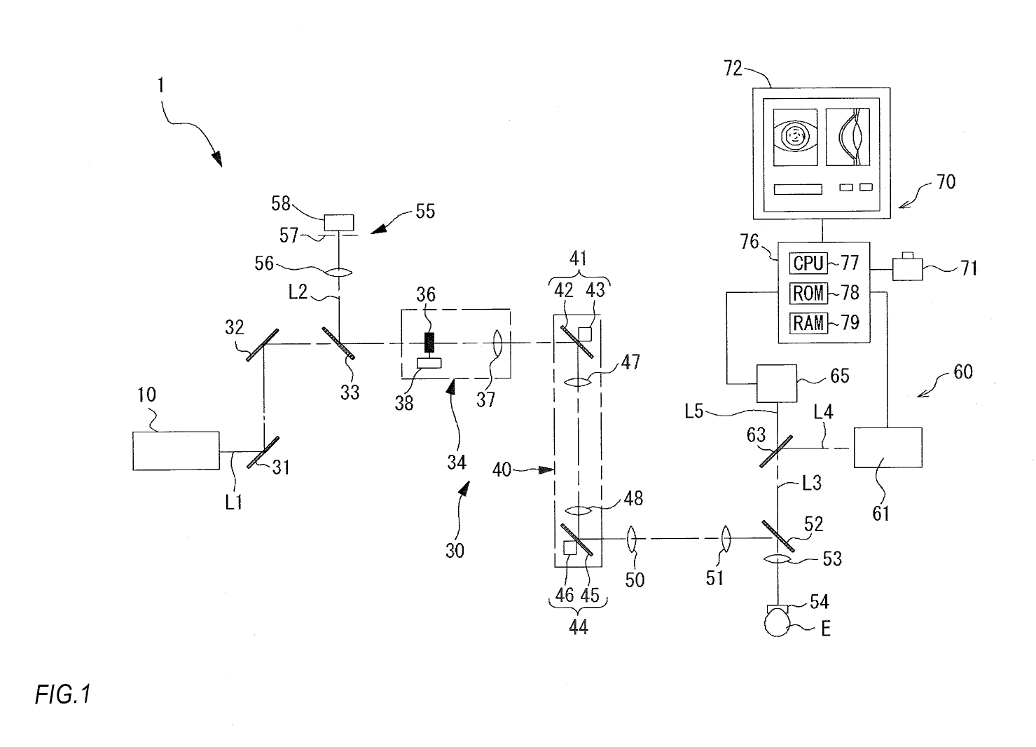

Hereinafter, a typical embodiment of the present disclosure will be described. First, the schematic configuration of a laser eye surgery apparatus 1 in the embodiment will be described with reference to FIG. 1. The following description will be given on the assumption that a direction of the visual axis of a patient's eye E is a Z-axis direction, a horizontal direction is an X-axis direction, and a vertical direction is a Y-axis direction. In the drawings, each lens, each mirror, or the like is illustrated by one member. However, each lens, each mirror, or the like may be formed of multiple optical components.

### <Entire Configuration>

The laser eye surgery apparatus 1 in the embodiment is used to treat a tissue (at least one of the cornea, the crystalline lens, and the like) of the patient's eye. The laser eye surgery apparatus 1 in the embodiment includes a laser device 10; a scanner 30; an objective lens 53; a position detecting unit 55; an observation and image capturing unit 60; an operation unit 70; and a control unit 76.

### <Laser Device>

The laser device 10 repeatedly emits multiple laser pulses. In the embodiment, the laser pulses emitted by the laser device 10 are used to cut and fragment a transparent tissue by causing the transparent tissue to photodisrupt. More specifically, in the embodiment, the laser pulses are used to induce plasma in the transparent tissue via non-linear interaction. The non-linear interaction is an interaction between light and a substance, and is an action in which a response appears non-proportionally to the intensity of light (that is, the density of photons). The laser eye surgery apparatus 1 in the embodiment causes multiphoton absorption to occur at a concentration position by concentrating (focusing) the laser pulses in the transparent tissue of the patient's eye E. The probability of the occurrence of the multiphoton absorption is not proportional to the intensity of light, and is non-linear. When an excited state occurs due to the multiphoton absorption, plasma is generated in the transparent tissue, thereby causing photodisruption. The induced plasma may be plasma which accompanies or does not accompany plasma emission.

As an example, the pulse width of the laser pulses emitted to the patient's eye by the laser device 10 may be 10 femtoseconds or greater and 1 nanosecond or less. In the embodiment, the laser pulses with a pulse width of 10 femtoseconds or greater and 10 picoseconds or less are exemplarily used. The laser device 10 will be described in detail later.

### <Scanner>

The scanner 30 scans the concentration position of each laser pulse concentrated by the objective lens 53 (to be described in detail later) by scanning the concentration position of each laser pulse emitted from the laser device 10. That is, the scanner 30 moves the concentration position of the laser pulses to a target position. The scanner 30 in the embodiment includes a Z scanner 34 and an X-Y scanner 40.

The Z scanner 34 in the embodiment includes a concave lens 36; a convex lens 37; and a driver 38. The driver 38 moves the concave lens 36 along an optical axis L1. When the concave lens 36 is moved, the diverging state of a beam passing through the concave lens 36 is changed. As a result, the concentration position (laser spot) of the laser pulses is moved in the Z-axis direction.

The X-Y scanner 40 in the embodiment includes an X scanner 41; a Y scanner 44; and lenses 47 and 48. The X scanner 41 scans laser pulses in the X-axis direction by swinging a galvanometer mirror 42 using a driver 43. The Y scanner 44 scans laser pulses in the Y-axis direction by swinging a galvanometer mirror 45 using a driver 46. The lenses 47 and 48 work in conjunction with two galvanometer mirrors 42 and 45.

Mirrors 31 and 32, and a hole mirror 33 are provided between the laser device 10 and the Z scanner 34. The mirrors 31 and 32 guide the laser pulses emitted by the laser device 10. The hole mirror 33 sets the optical axis L1 of the laser pulses to be coincident with an optical axis L2 of the position detecting unit 55 (to be described later). Lenses 50 and 51, and a beam combiner 52 are provided between the X-Y scanner 40 and the objective lens 53. The laser pulses are relayed through the lenses 50 and 51. The beam combiner 52 sets the optical axis L1 of the laser pulses to be coincident with an optical axis L3 of the observation and image capturing unit 60 (to be described later).

The configuration of the scanner 30 can be appropriately changed. For example, the lenses 47 and 48 between the X scanner 41 and the Y scanner 44 may be omitted. The laser eye surgery apparatus 1 may scan the laser pulses in the X-axis direction and the Y-axis direction using an acousto-optic modulator (AOM) or an acousto-optic device (AOD) polarizing the laser pulses, instead of the galvanometer mirrors 42 and 45. The laser pulses may be scanned in one direction by multiple elements. The laser pulses may be scanned by a resonant scanner, a polygon mirror, or the like. The Z scanner 34 may be positioned on a downstream side of the X-Y scanner 40, or the Z scanners 34 may be positioned on both of an upstream side and the downstream side of the X-Y scanner 40. Multiple Z scanners may be mounted on the upstream side or the downstream side of the X-Y scanner 40. The movement of the objective lens 53 in an optical axis direction allows the laser pulses to be scanned in the Z-axis direction. Other changes can also be added to the scanner 30.

### <Objective Lens>

The objective lens 53 is provided on an optical path between the scanner 30 and the patient's eye E. The objective lens 53 concentrates the laser pulses, which has passed through the scanner 30, on a tissue of the patient's eye E. In the embodiment, the laser pulses emitted from the objective lens 53 are concentrated on the tissue of the patient's eye E via a liquid immersion interface 54. The liquid immersion interface 54 may adopt a structure in which a cup suctioned and fixed to the patient's eye E is filled with liquid. An interface mounted on the patient's eye E is not limited to the liquid immersion interface 54. A contact lens attached to the patient's eye E may be used instead of the liquid immersion interface 54.

### <Position Detecting Unit>

The position detecting unit 55 is used to detect the position of the patient's eye E with respect to the scanner 30. The laser eye surgery apparatus 1 in the embodiment associates the concentration position of the laser pulses with a tomographic image (to be described in detail later) by detecting the position of the patent's eye E with respect to the scanner 30. Control data used to control the scanner 30 and the like can be set by associating the concentration position with the tomographic image.

In the embodiment, a portion of the optical system, through which the laser pulses pass, also serves as an optical system of the position detecting unit 55. The position detecting unit 55 includes the hole mirror 33; a concentration lens 56; an aperture plate 57; and a light receiving element 58. The hole mirror 33 transmits light incident to the center of the hole mirror 33, and reflects light, which is reflected by the patient's eye E, along the optical axis L2. The concentration lens 56 concentrates the light, which is reflected by the hole mirror 33, on an aperture of the aperture plate 57. The aperture plate 57 is a confocal aperture plate having an aperture at the center thereof. The aperture of the aperture plate 57 is disposed to work in conjunction with the concentration position (laser spot) of the laser pulses in the patient's eye E. The light receiving element 58 receives light passing through the aperture of the aperture plate 57. When the position of the patient's eye E is detected, the laser eye surgery apparatus 1 in the embodiment adjusts the output of laser light emitted from the laser device 10 in order for the laser light not to cause photodisruption at the concentration position. The laser eye surgery apparatus 1 causes the light receiving element 58 to receive light reflected from the patient's eye E while moving the concentration position three-dimensionally using the scanner 30.

The configuration used to detect the position of the patient's eye E with respect to the scanner 30 can be appropriately changed. For example, instead of the hole mirror 33, a polarizing beam splitter may be used to separate irradiating light from reflected light. The position detecting unit 55 may also be omitted. The laser eye surgery apparatus 1 may irradiate a sample substance or the like with the laser pulses, and detect an actual concentration position in the sample substance or the like via a tomographic image (to be described later).

### <Observation and Image Capturing Unit>

An operator observes the patent's eye E, and captures an image of a tissue, that is, a treatment target using the observation and image capturing unit 60. As an example, the observation and image capturing unit 60 in the embodiment includes an OCT unit 61 and a front observation unit 65. The beam combiner 52 sets the optical axis L3 of the observation and image capturing unit 60 to be coaxial with the optical axis L1 of the laser pulses. The optical axis L3 is split into an optical axis L4 of the OCT unit 61 and an optical axis L5 of the front observation unit 65 by a beam combiner 63.

The OCT unit 61 acquires a tomographic image of a tissue of the patient's eye E using optical coherence technology. Specifically, the OCT unit 61 in the embodiment includes a light source; a light splitter; a reference optical system; a scanner; and a detector. The light source emits light required to acquire a tomographic image. The light splitter splits the light emitted from the light source into reference light and measurement light. The reference light is incident to the reference optical system, and the measurement light is incident to the scanner. The reference optical system is configured to change the difference in an optical path length between the measurement light and the reference light. The scanner scans the measurement light on the tissue two-dimensionally. The detector detects a state of coherence between measurement light reflected by the tissue and the reference light passing through the reference optical system. The laser eye surgery apparatus 1 acquires information regarding the depth of the tissue by scanning the measurement light, and detecting a state of coherence between the reflected measurement light and the reference light. The laser eye surgery apparatus 1 acquires a tomographic image of the tissue based on the acquired information regarding the depth. The laser eye surgery apparatus 1 in the embodiment associates the concentration position of the laser pulses with the tomographic image of the patient's eye E captured prior to an operation. As a result, the laser eye surgery apparatus 1 is capable of preparing control data used to control a laser pulse irradiating operation (for example, the operations of the drivers 38, 43, and 46), based on the tomographic image. It is possible to adopt various configurations as the configuration the OCT unit 61. Any one of an SS-OCT, an SD-OCT, a TD-OCT, and the like may be adopted as the OCT unit 61.

The front observation unit 65 acquires a front image of the patient's eye E. The front observation unit 65 in the embodiment captures an image of the patient's eye E irradiated with visible light or infrared light, and displays the captured image on a monitor 72 (to be described later). The operator can observe the front of the patient's eye E by watching the monitor 72.

The configuration of the observation and image capturing unit 60 can also be appropriately changed. For example, the observation and image capturing unit 60 may adopt at least one of a configuration in which an image of the patient's eye E is captured using the Scheimpflug principle, a configuration in which an image of the patient's eye E is captured using ultrasonic waves, and the like.

### <Operation Unit>

The operation unit 70 receives various instructions input from the operator. As an example, the operation unit 70 in the embodiment includes an operation portion 71 with various operation buttons, and a touch panel provided on a surface of the monitor 72. The operation unit 70 may also adopt other configuration elements such as a joystick, a keyboard, and a mouse. The monitor 72 is capable of displaying various images such as a front image of the patient's eye E, a tomographic image of a tissue, and various operation menus.

### <Control Unit>

The control unit 76 includes a CPU 77; a ROM 78; a RAM 79; a non-volatile memory (not illustrated); and the like. The CPU 77 is in charge of various types of control (the control of the laser device 10, the control of the scanner 30, and the like) of the laser eye surgery apparatus 1. The ROM 78 stores various programs required to control the operation of the laser eye surgery apparatus 1, initial values, and the like. The RAM 79 temporarily stores various items of information. The non-volatile memory is a non-transitory storage medium capable of holding stored contents even if the supply of electrical power is shut off.

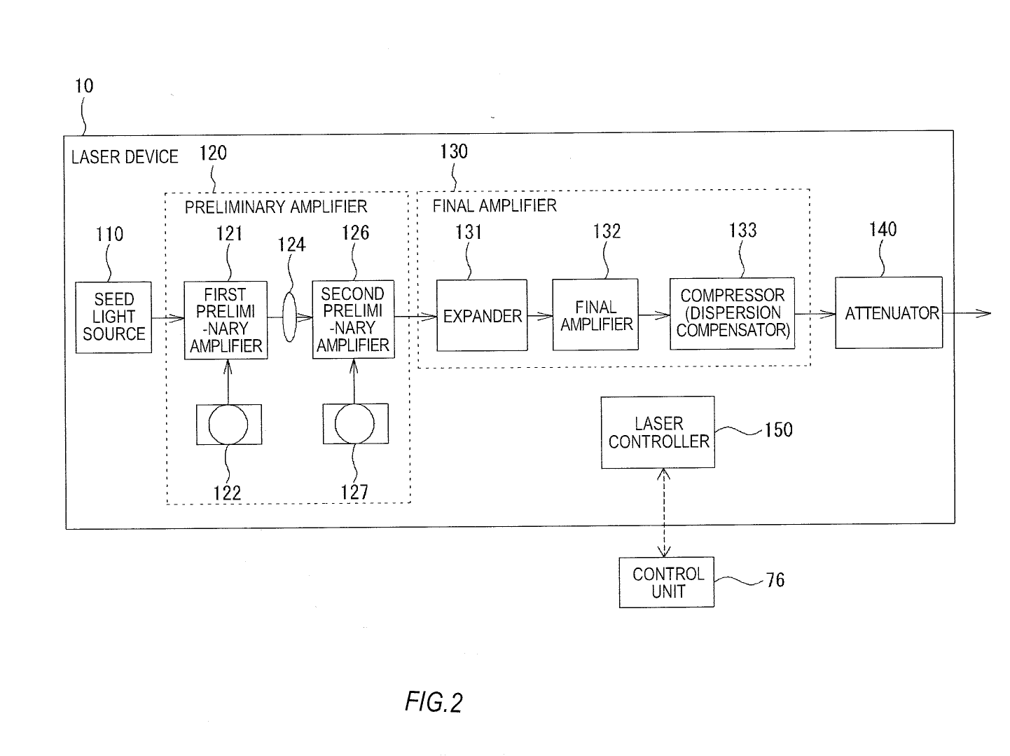

The laser device 10 in the embodiment includes a laser controller 150 (to be described later with reference to FIG. 2) controlling the emission of the laser pulses from the laser device 10, which will be described in detail later. The control unit 76 sends signals to and receives signals from the laser controller 150, and controls the emission of the laser pulses to the patient's eye E in collaboration with the laser controller 150. That is, in the embodiment, the control unit 76 and the laser controller 150 control the emission of the laser pulses. However, the configuration of the controller controlling the emission of the laser pulses can be appropriately changed. For example, the laser controller 150 may not be provided, and the control unit 76 may be fully in charge of control. Another controller may control the emission of the laser pulses.

### <Configuration of Laser Device>

The schematic configuration of the laser device 10 will be described with reference to FIG. 2. As illustrated in FIG. 2, the laser device 10 in the embodiment includes a seed light source 110; a preliminary amplifier 120; a final amplifier 130; an attenuator 140; and the laser controller 150.

### <Seed Light Source>

The seed light source 110 repeatedly generates a seed laser pulse (seed light) according to a repetition frequency determined by the controllers (in the embodiment, the control unit 76 and the laser controller 150). Particularly, the seed light source 110 in the embodiment is a gain-switched seed light source.

Hereinafter, the principle of generation of a seed laser pulse will be described. Examples of a method of generating the laser pulses include a mode synchronization (mode locking) method and a gain switching method (may be referred to as a Q switching method).

The mode synchronization method is a method of generating a laser pulse train at a constant repetition frequency by fixing a phase between longitudinal modes of laser light oscillating in multiple modes. The repetition frequency of a seed light source driven by the mode synchronization method is determined by the resonator length of laser light. Accordingly, the seed light source driven by the mode synchronization method used to generate an ultrashort pulse in the related art is not capable of changing the repetition frequency of a generated seed laser pulse. Also, when the seed light source is used according to the mode synchronization method, the filtering out of a non-amplified seed laser pulse, or the filtering out of a portion of multiple amplified laser pulses occurs, thereby resulting in a change in the repetition frequency of the laser pulses emitted to the patient's eye E. However, since it is necessary to filter out a portion of the multiple laser pulses at the same time intervals in this method, the repetition frequency is changed only in a stepwise manner. The filtered-out laser pulses are wasted.

In contrast, the gain switching method is a method of extracting the laser pulses by controlling the gain of a resonator. The gain-switched seed light source 110 is capable of linearly (continuously) changing the repetition frequency of a generated seed laser pulse. Accordingly, the laser device 10 in the embodiment is capable of appropriately changing the repetition frequency of the laser pulses (that is, laser pulse emitted to the patient's eye E) emitted to the outside of the laser device 10 by changing the repetition frequency of a seed laser pulse generated by the seed light source 110.

Various types of gain-switched light sources can be used as the seed light source 110. As an example, in the embodiment, a semiconductor laser is used as the seed light source 110. In this case, the operator can appropriately perform an operation for the patient's eye E using the laser eye surgery apparatus 1. A microchip laser can also be used as the seed light source 110. In this case, the operator appropriately treats the patient's eye E using the low-cost seed light source 110.

As an example, the seed light source 110 in the embodiment repeatedly generates a seed laser pulse with a pulse width of 10 femtoseconds or greater and 10 picoseconds or less. In this case, the operator can accurately treat a transparent tissue using an ultrashort pulse. However, even if the pulse width is 10 femtoseconds or greater and 1 nanosecond or less, treatment of the transparent tissue by photodisruption can be performed.

Multiple seed laser pulses (may also be referred to as a seed laser pulse train) generated by the seed light source 110 may be amplified and emitted to the outside of the laser device 10 without being filtered out. Naturally, a portion of a seed laser pulse train before amplification, or a portion of an amplified laser pulse train can also be filled out. When the amplified laser pulses are filtered out, a device (an acousto-optic device, a Pockels cell, or the like) for filtering out laser pulses may be disposed either inside or outside of the laser device 10.

### <Preliminary Amplifier>

The preliminary amplifier 120 receives and amplifies a seed laser pulse generated by the seed light source 110. A seed laser pulse has low energy. Accordingly, also, when a seed laser pulse with a pulse width in the order of femtoseconds is amplified, a probability of damage to the optical system of the preliminary amplifier 120 caused by self-focusing during amplification is low. Accordingly, various types of amplifying mechanisms can be used as the preliminary amplifier 120.

The preliminary amplifier 120 in the embodiment includes a first preliminary amplifier 121; a first excitation light source 122; a magnifying lens 124; a second preliminary amplifier 126; and a second excitation light source 127. Each of the first preliminary amplifier 121 and the second preliminary amplifier 126 is a multi-pass amplifier. Each of the first preliminary amplifier 121 and the second preliminary amplifier 126 contains an amplifying medium. A medium matched to the wavelength of a seed laser pulse may be used as the amplifying medium. The first excitation light source 122 and the second excitation light source 127 excite the amplifying media by irradiating the amplifying media, which are contained in the corresponding amplifiers, with excitation light. The excited amplifying media amplify an incident laser pulse, and emit the amplified laser pulse. The magnifying lens 124 is provided between the first preliminary amplifier 121 and the second preliminary amplifier 126, and increases the diameter of the laser pulses emitted to the second preliminary amplifier 126 from the first preliminary amplifier 121. The preliminary amplifier 120 may be a bulk type, or multiple optical fiber amplifiers may be used as the preliminary amplifier 120. A chirped pulse amplifier (to be described later) can also be used as the preliminary amplifier 120. Amplification stages may be appropriately set.

### <Final Amplifier>

The final amplifier 130 receives the laser pulses amplified by the preliminary amplifier 120, and amplifies energy of the laser pulse to an energy level greater than or equal to energy of the laser pulse emitted to the patient's eye E. Accordingly, since the laser eye surgery apparatus 1 in the embodiment includes the preliminary amplifier 120 and the final amplifier 130, even if the seed light source 110 generating a seed laser pulse having low energy is used, the operator can appropriately treat the patient's eye using the laser eye surgery apparatus 1.

As described above, the seed light source 110 in the embodiment generates a seed laser pulse with a pulse width of 10 femtoseconds or greater and 10 picoseconds or less. Since the pulse width is small, when an amplifying mechanism, for example, a master oscillator power amplifier (MOPA), is used as the final amplifier, the intensity of light may be excessively increased due to self-focusing while the seed laser pulse is amplified by the final amplifier. Accordingly, the optical system of the final amplifier may be damaged. As a result, in the embodiment, a chirped pulse amplifier is used as the final amplifier 130.

Specifically, the final amplifier 130 in the embodiment includes an expander 131; a final amplifier 132; and a compressor 133. The expander 131 expands the pulse width of the laser pulse received from the preliminary amplifier 120. The expander 131 in the embodiment expands the pulse width by applying a chirp, which is changed according to the frequency, to the laser pulses having a spectral width. At least one of a diffraction grating, a volume Bragg grating, a chirp mirror, and the like may be used as the expander 131. The final amplifier 132 amplifies the laser pulse, the pulse width of which is expanded by the expander 131. Since the laser pulse amplified by the final amplifier 132 has an expanded pulse width, the laser pulse has low peak power compared to the laser pulses, the pulse width of which is not expanded. Accordingly, damage to the optical system is unlikely to occur. It is possible to adopt various configurations as the configuration of the final amplifier 132. As an example, a regenerative amplifier, which amplifies the laser pulses while the laser pulse passes through multiple mirrors, is used as the final amplifier 132 in the embodiment.

### <Compressor/Dispersion Compensator>

The compressor 133 compresses the pulse width of the laser pulse amplified by the final amplifier 132. In the embodiment, the compressor 133 compresses the laser pulse by applying a chirp, which is opposite to the chirp applied by the expander 131, according to the frequency. At least one of a diffraction grating, a volume bragg grating, a chirp mirror, a prism pair, and the like may be used as the compressor 133.

The compressor 133 in the embodiment serves as a dispersion compensator compensating dispersion which is applied to the laser pulse by an element (for example, an amplifier) on an upstream side of the compressor 133 on the optical path. As a result, a change in the pulse width of the laser pulse is compensated. Since the compressor 133 also serves as the dispersion compensator, the amplification and the dispersion compensation of ultrashort-pulse laser light are performed using a simple configuration.

Particularly, the laser eye surgery apparatus 1 in the embodiment linearly changes the repetition frequency of a seed laser pulse generated by the seed light source 110. When the repetition frequency is changed, the amount of dispersion applied to the laser pulse by the amplifiers 121, 126, and 132 may be changed. In this case, the dispersion compensator in the embodiment changes the amount of compensated dispersion according to the repetition frequency of the laser pulse. As a result, even if the repetition frequency is changed, the laser pulses with an appropriate pulse width are emitted to the patient's eye E. Various methods can be adopted as a method of changing the amount of compensated dispersion. For example, the amount of compensated dispersion is changed by changing at least one of the position and the angle of an optical element included in the dispersion compensator. The laser eye surgery apparatus 1 may include a dispersion compensator separate from the compressor 133 so as to compensate dispersion applied to the laser pulses. In this case, the position of the dispersion compensator can be appropriately set.

As described above, the laser device 10 in the embodiment includes the amplifiers (the preliminary amplifier 120 and the final amplifier 130). Accordingly, even if a seed laser pulse generated by the seed light source has low energy, the laser eye surgery apparatus 1 is capable of irradiating the patient's eye with the laser pulses having appropriate energy.

### <Attenuator>

When energy (the amount of amplification) per unit time applied to the amplifiers 121, 126, and 132 is changed, the pulse width and the waveform of each laser pulse may be changed. Accordingly, the controllers in the embodiment change the repetition frequency while constantly maintaining the energy per unit time applied to the amplifiers 121, 126, and 132. In this case, when the repetition frequency is decreased, energy of each laser pulse is increased, and when the repetition frequency is increased, energy of each laser pulse is decreased. The laser eye surgery apparatus 1 in the embodiment includes the attenuator 140 adjusting energy of the laser pulse amplified by the amplifiers 120 and 130. The controllers control the attenuator 140 such that the laser pulses having appropriate energy are emitted to the patient's eye E even if the repetition frequency is changed. When the attenuator 140 is provided, the position of the attenuator 140 can be appropriately set. For example, the attenuator 140 may be provided outside of the laser device 10.

The laser eye surgery apparatus 1 may adjust the amount of amplification by the amplifiers 121, 126, and 132 instead of using the attenuator 140, or while controlling the attenuator 140. In this case, the pulse width and the waveform of the laser pulse may be changed. Accordingly, the laser eye surgery apparatus 1 may suppress a change in the pulse width by compensating dispersion according to the amount of amplification using the dispersion compensator.

### <Laser Controller>

The laser controller 150 controls the emission of the laser pulses from the laser device 10. Specifically, the laser controller 150 in the embodiment is electrically connected to the seed light source 110, the preliminary amplifier 120, the final amplifier 130, and the attenuator 140, and sends signals to and receives signals from the control unit 76 (refer to FIG. 1) of the laser eye surgery apparatus 1. The laser controller 150 controls the emission of the laser pulses to the patient's eye E in collaboration with the control unit 76. For example, when a signal specifying a repetition frequency is received from the control unit 76, the laser controller 150 performs control such that the seed light source 110 generates a seed laser pulse at the repetition frequency specified by the signal. In addition, when a signal specifying energy of the laser pulses is received from the control unit 76, the laser controller 150 controls the seed light source 110, the preliminary amplifier 120, the final amplifier 130, and the attenuator 140 such that the laser pulses having energy specified by the signal are emitted to the outside of the laser device 10. A microcomputer including a processor, a memory, and the like can be used as the laser controller 150.

In order for ultrashort-pulse laser light to be emitted to the patient's eye E, it is considered that the pulse width of the laser pulses is compressed to be smaller than the pulse width of a seed laser pulse generated by the seed light source 110, and the compressed laser pulse is emitted from the laser device 10. However, when the spectral width of the laser pulse is not larger than the spectral width of the seed laser pulse, in many cases, the compressing of the pulse width becomes difficult. In contrast, the laser device 10 in the embodiment is not required to have a configuration (for example, a configuration in which the spectral width is increased by self-phase modulation) in which the spectral width of the laser pulses is increased. That is, the laser pulses with a spectral width smaller than or equal to the spectral width of a seed laser pulse are emitted to the outside by the laser device 10 in the embodiment. Accordingly, the laser eye surgery apparatus 1 is capable of emitting an appropriate laser pulse to the patient's eye E using a simple configuration. The expression “spectral width smaller than or equal to the spectral width of a seed laser pulse” also includes a case in which the spectral width of the laser pulses is unintentionally increased to be greater than the spectral width of a seed laser pulse in a process of amplifying the laser pulse.

### <Repetition Frequency Changing Process>

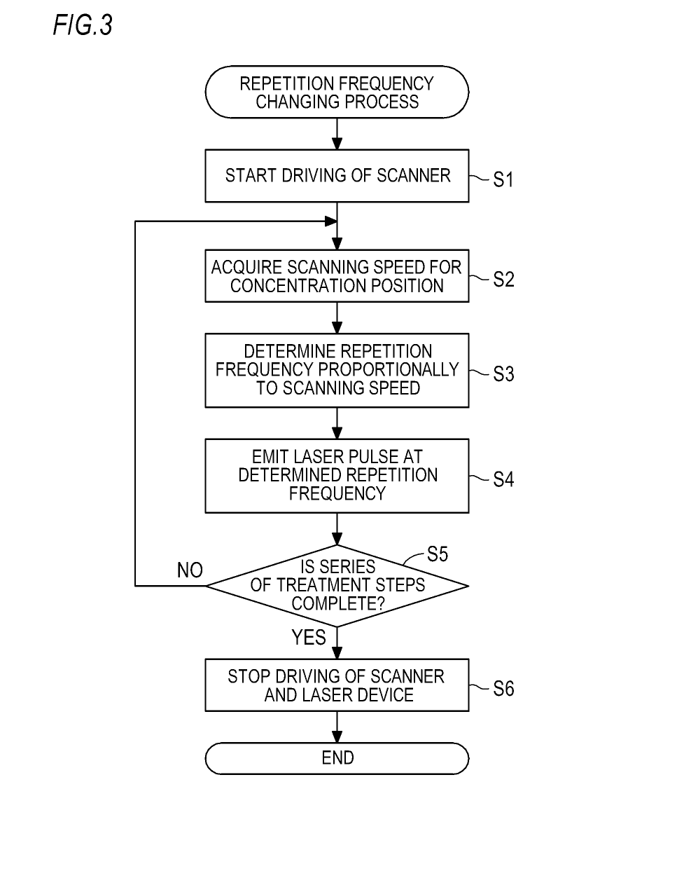

A repetition frequency changing process executed by the laser eye surgery apparatus 1 in the embodiment will be described with reference to FIG. 3. The laser eye surgery apparatus 1 in the embodiment executes the repetition frequency changing process such that the repetition frequency of the laser pulses emitted from the laser device 10 is changed according to a scanning speed at which a concentration position is scanned by the scanner 30.

The scanning speed for the concentration position may be affected and changed by performance of the scanner 30. When a scanning direction is inverted, the scanning speed can be decreased compared to when the concentration position is scanned straightly. When the concentration position is helically scanned, the scanning speed in a central portion of a helix is likely to be decreased compared to the scanning speed on the outside in the helix. When the repetition frequency of the laser pulses is the same before and after the scanning speed is changed, the space between adjacent concentration positions is not constant, and the quality of treatment may be decreased. The laser eye surgery apparatus 1 in the embodiment changes the repetition frequency of the laser pulses according to the scanning speed for the concentration position. As a result, the space between adjacent concentration positions easily becomes uniform, and a decrease in the quality of treatment is suppressed. The laser eye surgery apparatus 1 in the embodiment is also capable of appropriately linearly changing the repetition frequency of the laser pulses with respect to a linear change in the scanning speed. Accordingly, the operator can easily and appropriately treat the patient's eye E using the laser eye surgery apparatus 1 compared to when the repetition frequency is changed in a stepwise manner. The “linear change” represents a continuous linear change, and is not limited to a straight change.

When an instruction indicating start of treatment of the patient's eye E using the laser pulses is input via the operation portion 71 or the like, the repetition frequency changing process illustrated in FIG. 3 is executed by the CPU (processor) 77 of the control unit 76. The CPU 77 executes the repetition frequency changing process illustrated in FIG. 3 according to the program stored in the ROM 78 or the non-volatile memory.

First, the CPU 77 starts the driving of the scanner 30 according to drive data prepared in advance (S1). The CPU 77 acquires a scanning speed for a concentration position (S2). In the embodiment, the scanning speed for the concentration position is a scanning speed for a three-dimensional concentration position in the patient's eye E. Subsequently, the CPU 77 determines the repetition frequency of the laser pulses, which is emitted from the laser device 10, proportionally to the scanning speed acquired in step S2 (S3). As described above, the laser device 10 in the embodiment is capable of linearly changing the repetition frequency. Accordingly, the CPU 77 is capable of more appropriately disposing multiple concentration positions by linearly changing the repetition frequency according to a change in the scanning speed. The CPU 77 may not set the scanning speed to be perfectly proportional to the repetition frequency.

Subsequently, the CPU 77 performs controls such that the laser device 10 emits the laser pulses at the repetition frequency determined in step S2 (S4). In the embodiment, the control unit 76 and the laser controller 150 collaboratively control the emission of the laser pulse from the laser device 10. Accordingly, in step S4, a signal specifying the repetition frequency determined in step S2 is sent to the laser controller 150 by the CPU 77. The laser controller 150 makes the laser device 10 emit the laser pulses at the specified repetition frequency by making the seed light source 110 generate a seed laser pulse at the repetition frequency specified by the signal.

The CPU 77 determines whether or not a series of treatment steps determined by the drive data is complete (S5). When the series of treatment steps is not complete (NO: S5), the process returns to step S2, and steps S2 to S5 are repeated. When the series of treatment steps is complete (YES: S5), the driving of the scanner 30 and the laser device 10 is stopped (S6), and the process ends.

As described above, the laser eye surgery apparatus 1 in the embodiment includes the laser device 10, the scanner 30, and the controllers. The laser device 10 includes the gain-switched type seed light source 110 having a variable repetition frequency. The laser device 10 changes the repetition frequency of the laser pulses emitted to the outside by changing the repetition frequency of the seed light source 110. The controllers change the repetition frequency of the laser pulses, which is emitted to the patient's eye E from the laser device 10, according to a scanning speed at which the concentration position is scanned by the scanner 30. In this case, the laser eye surgery apparatus 1 is capable of changing the repetition frequency of the laser pulse in a stepwise manner or linearly (continuously). Since the laser eye surgery apparatus 1 is capable of changing the repetition frequency without filtering out a portion of a laser pulse train, energy efficiency is good. Since the repetition frequency of the laser pulse is changed according to the scanning speed for the concentration position, the space between adjacent concentration positions easily becomes uniform. Accordingly, the operator can accurately treat the patient's eye E within a short amount of time by appropriately changing the repetition frequency of the laser pulses from the laser eye surgery apparatus 1 in the embodiment. The configuration including a device (for example, acousto-optic device) for filtering out a portion of a laser pulse train is not a prerequisite for changing the repetition frequency.

The contents in the embodiment are merely exemplified. Accordingly, the contents in the embodiment can be changed. For example, the seed light source 110 in the embodiment generates a seed laser pulse with a pulse width of 10 femtoseconds or greater and 10 picoseconds or less. Accordingly, a chirped pulse amplifier is used as the final amplifier 130 so as to suppress damage to the optical system of the amplifying mechanism. However, when the laser pulses (for example, a laser pulse with a pulse width of 10 picoseconds or greater), which are unlikely to cause the occurrence of damage to the optical system, are used, an amplifying mechanism other than the chirped pulse amplifier may be adopted as the final amplifier.

Since the laser eye surgery apparatus 1 in the embodiment includes the preliminary amplifier 120 and the final amplifier 130, even if the seed light source 110 generating a seed laser pulse having low energy is used, the operator can appropriately treat the patient's eye using the laser eye surgery apparatus 1. In contrast, when a seed light source generating a seed laser pulse having high energy is used, the configuration of the amplifier may be changed. For example, the preliminary amplifier may be omitted, and only the final amplifier may be used. The laser device 10 may also be configured not to include the amplifier.

The expander 131 and the compressor 133 of the final amplifier 130 in the embodiment are separate components. However, the expander and the compressor may be integrally provided. For example, a volume Bragg grating may be used, and the laser pulses may be compressed by allowing the laser pulse to be incident to the volume Bragg grating from an incident direction opposite to an incident direction during expansion.

What is claimed is:

1. A laser eye surgery apparatus comprising:
a laser device which includes a gain-switched seed light source configured to repeatedly generate seed laser pulses with a pulse width of 10 femtoseconds or greater and 1 nanosecond or less and vary repetition frequency of the seed laser pulses according to a set repetition frequency, the laser device being configured to emit laser pulses based on the seed laser pulses generated by the gain-switched seed light source;
a condenser configured to condense the laser pulses emitted from the laser device in a transparent tissue of a patient's eye to cause photodisruption at concentration positions of the laser pulses in the transparent tissue;
a scanner configured to scan the concentration position of each of the laser pulses emitted from the laser device; and
a controller configured to control the gain-switched seed light source to change the repetition frequency of the seed laser pulses according to a scanning speed at which the concentration position is scanned by the scanner.

2. The laser eye surgery apparatus according to claim 1, wherein the laser pulses emitted from the laser device have a spectral width smaller than or equal to a spectral width of the seed laser pulse generated by the gain-switched seed light source.

3. The laser eye surgery apparatus according to claim 1, wherein the laser device includes an amplifier configured to amplify the seed laser pulses generated by the seed light source.

4. The laser eye surgery apparatus according to claim 3, wherein the amplifier of the laser device includes a preliminary amplifier configured to receive and amplify the seed laser pulses generated by the seed light source, and a final amplifier configured to receive the laser pulse amplified by the preliminary amplifier, and to amplify energy level of the amplified laser pulses to an energy level greater than or equal to energy of the laser pulse emitted to the patient's eye.

5. The laser eye surgery apparatus according to claim 3,
wherein the seed light source generates the seed laser pulses with a pulse width of 10 femtoseconds or greater and 10 picoseconds or less,
wherein the amplifier includes a chirped pulse amplifier, and
wherein the chirped pulse amplifier includes an expander configured to expand the pulse width of the laser pulses, an amplifier configured to amplify the laser pulses expanded by the expander, and a compressor configured to compress the pulse width of the laser pulses amplified by the amplifier.

6. The laser eye surgery apparatus according to claim 5, wherein the compressor serves as a dispersion compensator configured to compensate dispersion of the laser pulses.

7. The laser eye surgery apparatus according to claim 3, wherein the laser device includes an attenuator configured to adjust energy of the laser pulses amplified by the amplifier.

8. The laser eye surgery apparatus according to claim 1, wherein the laser device includes a dispersion compensator configured to compensate dispersion of the laser pulses.

9. The laser eye surgery apparatus according to claim 1, wherein the seed light source of the laser device includes a semiconductor laser.

10. The laser eye surgery apparatus according to claim 1, wherein the seed light source of the laser device includes a microchip laser.
